(19) United States
(12) Patent Application Publication
(10) Pub. No.: US 2026/0091046 A1
(43) Pub. Date: Apr. 2, 2026

(54) Method For Treatment Of Pancreatic Cancer

(71) Applicant: Cannabis Bioscience International Holdings, Inc., Houston, TX (US)

(72) Inventors: Dante Picazo, Houston, TX (US); Stephanie Dona Hartmann, Caracas (VE); Rosangel Del Valle Andrades Fuentes, Caracas (VE); Nancy Paola Duarte Delgado, Bogota (CO); Nancy Rodrigues Das Fontes, Viseu (PT); Ana Maria Maldonado Vacca, Bogota (CO)

(73) Assignee: Cannabis Bioscience International Holdings, Inc., Houston, TX (US)

(21) Appl. No.: 18/905,128

(22) Filed: Oct. 2, 2024

(51) Int. Cl. A61K 31/00; A61K 9/48; A61K 9/50; A61K 31/015; A61K 31/353

(52) U.S. Cl. CPC A61K 31/658; A61K 9/4858; A61K 9/4866; A61K 9/50; A61K 31/015; A61K 31/353

(57) ABSTRACT

Aspects of the disclosure provide a formulation for treating advanced pancreatic cancer, comprising a combination of a cannabinoid, a terpene, and a flavonoid. To enhance bioavailability and targeted delivery, the formulation is incorporated into a nanoplatform, such as a nanoemulsion, optimizing absorption and delivery to cancer cells. The nanoparticle-based composition specifically targets advanced stages of pancreatic cancer, aiming to inhibit tumor progression and improve clinical outcomes.

### CROSS-REFERENCE TO RELATED APPLICATIONS

None.

### FIELD OF THE DISCLOSURE

Aspects of the disclosure provide for treating stage III and IV pancreatic cancer by orally administering compositions comprising phytocannabinoids in nanoemulsion capsules. Aspects of the disclosure exhibit antiproliferative, antimetastatic, and apoptosis-inducing activities, improving treatment outcomes.

### BACKGROUND INFORMATION

Pancreatic cancer (PC) is a highly aggressive malignancy with a significant mortality risk, predominantly affecting older adults, typically between 60 and 85 years old, although there is an increasing incidence among younger patients. Known for its rapid invasion of surrounding tissues and organs, pancreatic cancer often progresses without early symptoms, contributing to its status as one of the deadliest forms of cancer.

The origins of pancreatic cancer remain a contentious topic among members of the health care community. Some believe hypotheses suggesting acinar, ductal, or islet cells are potential progenitors for pancreatic cancer. What is known is that each hypothesis is evaluated based on existing literature and recent findings, highlighting the potential for phenotypical transdifferentiation among pancreatic cells such as ductal to islet, ductal to acinar, acinar to ductal, acinar to islet, islet to acinar, and islet to ductal transitions. To date, no conclusive proof for the origin of pancreatic cancer has been reached. Current discussions in the health care community lean towards islet cells as a likely origin of pancreatic tumors. These beliefs are supported by islet cells' robust ability to transdifferentiate into both pancreatic and non-pancreatic cell types. These cells have the presence of specialized carcinogen-metabolizing enzymes that are exclusive to such cells as well as the growth factor-rich microenvironment within islets.

Pancreatic cancer can be broadly classified into four categories: including adenocarcinoma, squamous cell carcinoma, colloid carcinoma, and neuroendocrine tumors. Of these categories, two major categories are present in a majority of cancers. These include exocrine pancreatic cancer, which includes adenocarcinoma, and neuroendocrine pancreatic cancer. Each category encompasses various types of cancer that can differ in their symptoms and prognosis.

The pancreas is vital for the human digestive system. The pancreas assists in food breakdown and sugar utilization for energy. When the pancreas malfunctions, numerous issues may develop, including digestive problems, diabetes, and even cancer.

Pancreatic adenocarcinoma, the most prevalent form of pancreatic cancer, constitutes approximately 95 percent of cases. In contrast, there exists another type known as pancreatic neuroendocrine tumors (pNETs), which can sometimes be mistaken for pancreatic adenocarcinoma, despite their distinct origins and characteristics. Scientific studies indicate that pancreatic adenocarcinoma arises from an abnormal proliferation of exocrine cells. These exocrine cells are responsible for producing digestive enzymes. Pancreatic neuroendocrine tumors develop from endocrine cells that regulate blood sugar levels by producing hormones like insulin. These fundamental differences underscore the diverse nature of pancreatic cancers, influencing diagnosis, treatment approaches, and patient outcomes accordingly.

Medical studies have found that smoking, alcohol, and chronic pancreatitis are considered high-risk factors. Recent studies have shown that abnormal metabolism of human microorganisms, blood type, glucose levels, and lipid levels are also important factors in the development of pancreatic cancer.

Unfortunately, the incidence of pancreatic cancer has increased over the last few years. While pancreatic cancer accounts for approximately 2 percent of all cancers; it is responsible for 5 percent of cancer related deaths. Thus, pancreatic cancer is a major contributor for mortality of humans, far exceeding its prevalence in the population. Complicating diagnosis and treatment regimes, most patients exhibit no obvious symptoms during the development and progression of the disease to advanced metastatic stages, where tumor cells become highly invasive.

The lack of early symptoms makes early diagnosis challenging, and pancreatic cancer has become one of the most lethal malignant tumors. Even after potential radical treatment, the majority of patients experience relapse, and the 5-year survival rate remains low. Typically, the 5-year survival rate is between 2 percent to 9 percent. Pancreatic ductal adenocarcinoma (PDAC) is the most common type of pancreatic cancer. Medical professionals look to risk factors to determine the possible susceptibility of an individual to pancreatic cancer. Chief among these risk factors include family history, pancreatitis, and diabetes. Despite the lethality of pancreatic cancer, there are currently no standardized screening programs worldwide for high-risk individuals. There is a long felt need for not only screening for pancreatic cancer, but also treatment of the symptoms as well as the cancer itself. As time moves forward, such needs grow greater, with no conventional treatment regimens capable of a solution.

As stated above, unfortunately, pancreatic cancer usually shows little or no symptoms until it has advanced and spread. Current medical studies show that most cases (up to 80 percent) are diagnosed at later, more difficult-to-treat, stages. The primary challenge lies in the difficulty of diagnosing pancreatic cancer early, as there are no specific, cost-effective, screening tests that can reliably detect the disease in asymptomatic individuals. Consequently, pancreatic cancer is often discovered at advanced stages when surgical removal is no longer viable, and the cancer has metastasized to other parts of the body. According to the Surveillance, Epidemiology, and End Results (SEER) database, for every 12.2 patients diagnosed per 100,000, 10.9 will die from pancreatic cancer, despite the best efforts of researchers and clinicians to improve survival outcomes.

Different researchers reported that pancreatic cancer is increasingly diagnosed at an advanced stage in younger patients. Another study has found that nearly 50 percent of patients aged 16 to 54 with pancreatic cancer are more likely to be diagnosed at an incurable stage, due to poor awareness, misdiagnosis, and delays in care. Some studies confirm that pancreatic cancer is frequently diagnosed at an advanced stage, potentially due to the aggressive nature of the tumor and the often non-specific symptoms in younger individuals. Studies indicate that patients with less common cancers, such as pancreatic cancer, are more likely to require three or more visits to their primary care physician before being referred to a specialist.

As noted above, approximately 90 percent of pancreatic cancers are classified as pancreatic ductal adenocarcinoma. Of the risk factors for pancreatic cancer previously described, familial predisposition is the chief contributor, due to susceptibility gene mutations, chronic pancreatitis, pancreatic cysts, and diabetes mellitus. Additional risk factors include, but are not limited to smoking or other exposure to air contaminants, alcohol abuse, obesity or metabolic syndrome, aging, and occupational exposure. Even among those eligible for early-stage resection, the 5-year survival rate is less than thirty one percent (31 percent). Recent advances in next-generation genome sequencing (NGS) have generated renewed optimism in the fields of diagnosis and treatment by enabling the identification of molecular alterations within an individual that drive pancreatic cancer progression. Sequencing data from NGS have shown that pancreatic cancer consists of highly heterogeneous tumors that often develop resistance to traditional chemotherapy and radiation therapy. Due to the resistance to traditional chemotherapy and radiation therapy, there is a need for early detection for effective treatment.

Early detection of pancreatic cancer is hindered by the absence of biomarkers and the deep anatomical location of the pancreas. Despite 70 percent of pancreatic ductal adenocarcinomas occurring in the pancreatic head, often leading to bile duct obstruction and jaundice, early diagnosis remains rare. Symptoms typically manifest in advanced stages, including abdominal pain, weight loss, anorexia, nausea, and vomiting. A significant challenge in biomarker development is that 80 percent of patients are diagnosed late; resulting in blood samples reflecting advanced disease stages. Currently, Carbohydrate Antigen 19-9 (CA19-9) is the sole FDA-approved biomarker for pancreatic ductal adenocarcinoma, yet this biomarker lacks sensitivity and specificity in early cancer stages.

Typical presenting symptoms of pancreatic cancer include abdominal or mid-back pain, obstructive jaundice, and weight loss. Weight loss can arise from anorexia, maldigestion from pancreatic ductal obstruction, and cachexia. Occasionally, pancreatic duct obstruction could result in attacks of pancreatitis. Deep and superficial venous thrombosis is not unusual and might be a presenting sign of malignant disease. Gastric-outlet obstruction with nausea and vomiting sometimes happens with more advanced disease. Less common manifestations include panniculitis and depression. About 25 percent of patients with pancreatic cancer have diabetes mellitus at diagnosis and roughly, another 40 percent have impaired glucose tolerance.

Phytocannabinoids are substances that have been highly regulated or excluded from medical care in certain countries. Phytocannabinoids may include, but not be limited to, Tetrahydrocannabinol (“THC”), tetrahydrocannabivarin (“THCV”), Cannabidiol (“CBD”), cannabigerol (“CBG”), limonene, and kaempferol. These substances are essentially unknown or new in the medical field. There currently is very little knowledge of the use of such substances in various areas of medicine. Such substances have been banned in many areas and as such they are not used in conventional medical treatments.

### Conventional Treatments for Pancreatic Cancer

The following conventional treatments are currently used in the treatment of pancreatic cancer:
Surgery: The only curative option for PC, classified based on respectability and often combined with neoadjuvant therapy, to improve outcomes.
Chemotherapy: Essential for both adjuvant and metastatic settings, with FOLFIRINOX and gemcitabine-based regimens being common.
Radiotherapy: Used mainly for locally advanced PC, with various forms including external beam radiation and brachytherapy.
Palliative Care: Important for managing symptoms and improving quality of life in advanced stages.
Targeted Therapy: Focuses on specific molecular targets; however, many targeted drugs have yet to show efficacy in PC.
Immunotherapy: Currently limited success, but ongoing research explores combinations with other treatments.
Microbial Therapy: Investigates the role of microbiota in regulating immune responses and treatment efficacy.

There is a need for a novel treatment of pancreatic cancer as the conventional therapies and treatments have shown ineffectiveness for large sections of the population.

There is a further need to provide a cost-effective treatment that can be used in a wide range of scenarios of pancreatic cancer.

There is a still further need to provide a treatment that does not need specialized training for health care individuals.

There is a further need to provide a treatment that may be used for young and older patients alike.

There is a further need to provide a treatment that may be used in a home setting or that may be administered in a home setting, thereby reducing the ever-increasing number of pancreatic cancer cases treated at hospitals.

### SUMMARY OF THE INVENTION

Aspects of the disclosure present a approach for the treatment of pancreatic cancer composition for the treatment of pancreatic cancer, comprising a) an active principle and b) stabilizing solubilizing agent, wherein the active principle comprising a cannabinoid, a terpene, and a flavonoid. Aspects of the disclosure further describe to a formulation that incorporates a cannabinoid, a terpene, and a flavonoid into a nanoplatform, preferably, a nanoemulsion in the form of nanoparticles. Particularly, the composition is a nanoemulsion formulation comprising nanoparticles. The formulation aims to effectively target pancreatic cancer cells, particularly in advanced stages. By combining the therapeutic potential of these compounds with the advantages of nanoemulsion delivery, this disclosure offers a promising strategy for improving outcomes in patients with metastatic or locally advanced pancreatic cancer.

Aspects of the disclosure also refer to a method for producing a formulation for pancreatic cancer treatment by incorporating cannabinoids, terpenes, and flavonoids into a nanoemulsion, which are then included in the final formulation. The aspects further describe to a pharmaceutical composition comprising a nanoemulsion carrying a combination of a cannabinoid, a terpene limonene, and a flavonoid kaempferol.

Additionally, the aspects of the disclosure describe a treatment method using the formulation to target pancreatic cancer in patients with stage III or IV pancreatic cancer, particularly those with primary tumors in the pancreatic head or multiple metastatic sites and pancreatic adenocarcinoma. The formulation is administered in a dosage regimen tailored to effectively target these advanced cancer stages and improve patient outcomes.

The aspects of the disclosure also refer to a method of treating metastatic or locally advanced pancreatic cancer in a human individual is disclosed. The method comprises administering to the human individual, an effective concentration of a composition comprising nanoparticles. Said nanoparticles comprising cannabinoids, terpenes, and flavonoids (THC, THCV, CBD, CBG, CBD, limonene, and kaempferol) wherein the individual is selected for treatment based on at least one of (i) having stage III pancreatic cancer, (ii) having stage IV metastatic cancer, (iii) having pancreatic cancer in a primary location in a pancreatic head, (iv) having more than 3 metastatic sites, and (v) having palliative care.

### BRIEF DESCRIPTIONS OF THE FIGURES

To provide a detailed understanding of the features mentioned above, the following description refers to specific examples, some of which are shown in the drawings. However, these drawings only represent typical examples and should not be seen as limiting, as other equally effective versions of the disclosure are possible.

FIG. 1. illustrates an overview of aspects of the disclosure relating to patients with stage III and IV pancreatic cancer. It highlights enhanced and broadened palliative care by suppressing tumor growth, reducing metastatic activity, and mitigating the aggressiveness of the disease.

FIG. 2. illustrates the effects and mechanism of action of aspects of the disclosure in pancreatic cancer cells, as well as its role in protecting and enhancing cellular response in healthy cells. The aspects described exhibit antiproliferative, antimetastatic, and antiangiogenic properties, indicating its ability to inhibit the growth of cancer cells, prevent metastasis, and reduce the formation of new blood vessels crucial for tumor growth.

FIG. 3. illustrates the treatment approach of aspects of the disclosure focusing on oral administration of a comprehensive formulation containing cannabinoids, terpenes, and flavonoids.

FIG. 4. illustrates the anti-cancer properties of cannabinoids THC, THCV, CBD, CBG, and CBC, the terpene limonene, and the flavonoid kaempferol.

To facilitate understanding, identical reference numerals have been used, where possible, to designate identical elements that are common to the figures (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood; however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments, and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers and/or sections, these elements, components, regions, layers and/or sections should not be limited by these terms. These terms may be only used to distinguish one element, components, region, layer or section from another region, layer, or section. Terms such as “first”, “second”, and other numerical terms, when used herein, do not imply a sequence or order unless clearly indicated by the context. Thus, a first element, component, region, layer, or section discussed herein could be termed a second element, component, region, layer, or section without departing from the teachings of the example embodiments.

When an element or layer is referred to as being “on”, “engaged to”, “connected to”, or “coupled to” another element or layer, it may be directly on, engaged, connected, coupled to the other element or layer, or interleaving elements or layers may be present. In contrast, when an element is referred to as being “directly on”, “directly engaged to”, “directly connected to”, or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the figures. Like elements in the various figures will be referenced with like numbers for consistency. In the following description, numerous details are set forth to provide an understanding of various embodiments and/or features. It will be understood; however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

### Definitions

The following definitions are for description only and do not form limiting concepts. The term “individual” refers to a mammal, including humans. An individual includes, but is not limited to, human, bovine, horse, feline, canine, rodent, or primate. In some embodiments, the individual is human. The term “individual” also includes human patients that are undergoing treatment for pancreatic cancer treatment.

As used herein, “treatment” or “treating” refers to an approach aimed at achieving beneficial or desired outcomes, including clinical results. For the purposes of the disclosure, these beneficial or desired clinical results encompass, but are not restricted to, the following possibilities related to pancreatic cancer: alleviating one or more symptoms caused by the disease, reducing the extent of pancreatic cancer, maintaining the disease at a stable level, preventing or delaying the spread of pancreatic cancer, preventing or delaying the recurrence of pancreatic cancer, slowing the progression of pancreatic cancer, and improving the disease condition. Other non-limiting possibilities exist, including achieving remission (partial or total) of pancreatic cancer, reducing the dosage of one or more other medications needed to treat pancreatic cancer, enhancing quality of life for pancreatic cancer patients, promoting weight gain in pancreatic cancer patients, and/or extending survival of pancreatic cancer patients. Additionally, “treatment” includes reducing the pathological consequences specifically associated with pancreatic cancer. The methods described herein encompass any one or more of these treatment aspects related to pancreatic cancer.

The term “dosage or dose” as used herein refers to the quantity of a compound or composition adequate for treating a specified disorder, condition, or disease, including alleviating, palliating, reducing, and/or delaying one or more of its symptoms. Specifically concerning pancreatic cancer, an effective dose is defined as the quantity necessary to induce tumor shrinkage and/or reduce the tumor growth rate (such as by suppressing tumor growth) or to prevent or delay other undesirable cell proliferation in pancreatic cancer. In certain scenarios, an effective amount is the quantity required to delay the onset of pancreatic cancer. Additionally, in specific instances, an effective amount is the quantity necessary to prevent or delay recurrence.

The term “effective dose” is an amount of substance that can be provided in one or more administrations. In the case of pancreatic cancer, the effective dose of the composition or formulation may: (i) reduce the number of pancreatic cancer cells; (ii) decrease tumor size; (iii) inhibit, slow to some extent, and in some instances halt, pancreatic cancer cell infiltration into peripheral organs; (iv) inhibit (i.e., slow to some extent, and preferably stop) tumor metastasis; (v) inhibit tumor growth; (vi) prevent or delay the occurrence and/or recurrence of tumors; (vii) alleviate to some extent one or more symptoms associated with pancreatic cancer; and/or (viii) disrupt (such as destroy) pancreatic cancer stroma.

The term “pancreas” is referred to as an organ located behind the stomach. This organ is useful in both the digestive and endocrine systems. The pancreas produces digestive enzymes that break down proteins, fats, and carbohydrates in the small intestine. These enzymes are transferred into the duodenum. An additional function of the pancreas is to produce hormones like insulin and glucagon, which regulate blood sugar levels. This dual role in nutrient absorption and glucose metabolism makes the pancreas crucial for overall bodily function.

The term “pancreatic cancer” refers to a malignant tumor that originates in the pancreas, includes adenocarcinoma, squamous cell carcinoma, colloid carcinoma, and neuroendocrine tumors. The term also includes squamous cell carcinoma, which develops in the squamous cells of the exocrine pancreas and is rare in prevalence. Pancreatic colloid carcinoma develops in the cells lining the ducts and the presence of extracellular mucin containing neoplastic cells. Neuroendocrine tumors arise from a type of cell called islet cells of Langerhans, which secrete one or more active peptides.

The term “pancreatic cancer” encompasses the most common pancreatic cancer that arises in the exocrine cells lining the pancreatic ducts, with adenocarcinoma being the predominant type.

The term “ductal pancreatic adenocarcinoma”, hereinafter “PDAC” is the most prevalent form of pancreatic cancer. PDAC is known for having an aggressive nature and poor prognosis due to often being diagnosed at an advanced stage. Originating in the pancreatic ducts, PDAC frequently presents with symptoms like jaundice, abdominal pain, and weight loss, in later stages.

The term “risk factors” in pancreatic cancer is any attribute, characteristic, or exposure that increases the likelihood of developing the disease. Non-limiting risk factors for developing pancreatic cancer include smoking, chronic pancreatitis, diabetes, obesity, and genetic predispositions.

The term “diagnosis” of pancreatic cancer typically involves a combination of medical history evaluation, physical examination, and various diagnostic tests. A diagnosis may occur with a single evaluation, physical examination, or diagnostic test. In non-limiting embodiments, these steps may be taken singularly or in any order. In some embodiments, imaging studies like computed tomography “CT” scans, magnetic resonance imaging “MRI”, and endoscopic ultrasound evaluations are commonly used to visualize the pancreas and detect tumors. Blood tests, including the CA 19-9 tumor marker, can aid in diagnosis but are not definitive on their own. A biopsy, often performed using endoscopic ultrasound-guided fine needle aspiration, may be used to confirm the presence of cancer cells.

The term “biomarker” is a measurable biological indicator that can be used to detect the presence of pancreatic cancer, assess a cancer stage or progression, predict treatment response, and/or monitor recurrence. Biomarkers may include proteins, genetic mutations, circulating tumor cells, or other molecules that are found in blood, tissue, or other bodily fluids. Biomarkers may play a crucial role in early detection and personalized treatment strategies by providing insights into the molecular characteristics of the cancer and potential response to therapies. Biomarkers may be used in guiding clinical decision-making, improving patient outcomes, and advancing research efforts aimed at better understanding and combating pancreatic cancer.

The term “main biomarkers in pancreatic cancer” include CA 19-9, a blood marker used for monitoring disease progression; mutations in genes like KRAS, TP53, and SMAD4, which influence cancer development and treatment responses; circulating tumor cells (CTCs) that indicate disease spread; and microRNAs (miRNAs) and DNA methylation patterns that reflect genetic alterations. These biomarkers play crucial roles in diagnosis, prognosis, and treatment decisions, guiding clinicians in tailoring therapies and monitoring patient responses effectively. The term “pharmacological treatment” refers to the use of medications and drugs, compounds, or formulations, to manage and treat the disease. Non-limiting embodiments include chemotherapy, targeted therapy, immunotherapy, and other pharmacological agents aimed at inhibiting tumor growth, shrinking tumors, relieving symptoms, and improving overall survival rates.

The term “treatment of pancreatic cancer” depends on the stage and overall health of the patient and may include surgery, chemotherapy, radiation therapy, and targeted therapy.

The term “stage” is defined as a determined extent and spread of the disease within the body. Staging categorizes pancreatic cancer based on factors such as the size of the tumor, the cancer's involvement with nearby tissues and organs, whether the cancer has spread to lymph nodes, as well as if it has metastasized to distant sites like the liver or lungs. This classification system, often using the TNM (Tumor, Nodes, Metastasis) system, helps healthcare providers assess the prognosis for patients and guide treatment decisions. For instance, early-stage cancer (I and II) typically indicates localized disease, potentially eligible for surgical removal or targeted therapies aimed at controlling the cancer's progression. Advanced stages (III and IV) indicate more extensive spread, often requiring systemic treatments like chemotherapy or radiation to manage symptoms and prolong survival.

“Stage III” pancreatic cancer is defined as a cancer that has advanced beyond the pancreas and nearby tissues, spreading extensively into surrounding blood vessels, lymph nodes, or other nearby organs. At this stage, the tumor may be large and have infiltrated nearby structures, making surgical removal challenging. Typically, stage III pancreatic cancer is characterized by an aggressive nature and increased likelihood of causing symptoms such as abdominal pain, jaundice, and weight loss. Treatment options for stage III pancreatic cancer often include a combination of chemotherapy, radiation therapy, and sometimes targeted therapies to shrink the tumor, alleviate symptoms, and potentially make surgery more feasible. The prognosis for stage Ill pancreatic cancer varies depending on factors such as the size and location of the tumor, response to treatment, and overall health of the patient.

The term “stage IV” pancreatic cancer is defined as the most advanced stage of the disease, where cancer cells have spread extensively beyond the pancreas to distant organs such as the liver, lungs, or peritoneal cavity. At this stage, the cancer is often widespread and difficult to treat with curative intent. Patients with stage IV pancreatic cancer typically experience significant symptoms including abdominal pain, jaundice, digestive problems, weight loss, and fatigue. Treatment aims to alleviate symptoms, improve quality of life, and prolong survival through a combination of chemotherapy, targeted therapy, and palliative care measures. Surgery is generally not an option due to the widespread nature of the disease. The prognosis for stage IV pancreatic cancer is usually poor, with a median survival time typically measured in months.

The term “palliative care” is defined as improving the quality of life of a patient by managing symptoms such as pain, nausea, and fatigue, and providing emotional and psychological support. Palliative care involves addressing physical discomfort, offering counseling, and assisting with nutritional needs. Palliative care also includes spiritual support and ensuring coordinated care among healthcare providers. This care can be provided alongside curative treatments or as the primary focus when treatments are no longer effective, prioritizing the patient's comfort and well-being.

The term “pancreatic cancer patients” refers to individuals diagnosed with pancreatic cancer, encompassing both those currently undergoing treatment and those managing the disease in stage III and IV. This term is inclusive of persons who have received a confirmed diagnosis of pancreatic cancer and are undergoing medical care, supportive therapies, or palliative interventions aimed at managing symptoms and improving quality of life.

The term “gene” refers to a specific sequence of DNA that encodes instructions for producing proteins or regulating cellular functions. Mutations or alterations in certain genes can contribute to the development and progression of pancreatic cancer.

The term “target genes” refer to specific genes that are altered or mutated, contributing to the development and progression of the disease. These genes often encode proteins involved in critical cellular processes such as cell growth, differentiation, and survival. The most targeted genes in pancreatic cancer include KRAS, TP53, CDKN2A, and SMAD4.

The term, “cannabinoids” is referred as chemical compounds found in the cannabis plant, including THC (tetrahydrocannabinol) and CBD (cannabidiol), known for their interactions with the human body's endocannabinoid system, which can influence various physiological processes.

The term “terpenes” is defined as organic compounds found in plants, including cannabis, that potentially offer therapeutic benefits, including anti-cancer properties. Research suggests that certain terpenes may contribute to anti-inflammatory, antioxidant, and potentially anti-cancer effects.

The term “flavonoids” is a diverse group of plant compounds known for their antioxidant and anti-inflammatory properties. They are found in various fruits, vegetables, and herbs, and play a role in cancer prevention and treatment. Some research suggests that certain flavonoids may inhibit the growth of cancer cells, reduce inflammation, and promote apoptosis (cell death) in cancer cells.

The term “endocannabinoid system” (ECS) describes a complex biological system composed of cannabinoid receptors, endocannabinoids (which are endogenous lipid-based neurotransmitters), and enzymes involved in their synthesis and degradation. This biological system plays a role in regulating various physiological processes, including mood, memory, pain sensation, appetite, and immune response. The ECS helps maintain homeostasis within the body by modulating these functions in response to internal and external stimuli.

The term “CB1 receptor”, short for cannabinoid receptor type 1, is a protein receptor found primarily in the central nervous system and peripheral tissues. The CB1 receptor plays a role in mediating the effects of cannabinoids, both endogenous (produced naturally within the body) and exogenous (from external sources like cannabis). Activation of CB1 receptors modulates various physiological processes including pain sensation, appetite, mood, and memory.

The term “CB2 receptor”, or cannabinoid receptor type 2, is a protein receptor predominantly found on immune cells and tissues throughout the body. The CB2 receptor plays a role in regulating immune function and inflammatory responses. CB2 receptors are activated by cannabinoids, influencing processes such as immune cell migration, cytokine release, and inflammation modulation. Unlike CB1 receptors, CB2 receptors are less abundant in the central nervous system, but are widely distributed in peripheral tissues, making them a significant target for therapeutic interventions aimed at immune-related disorders and inflammation.

The term “CBD”, or cannabidiol, is a non-psychoactive compound found in cannabis plants. It is known for its potential therapeutic effects, including reducing anxiety, pain, and inflammation. In cancer, CBD is being studied for its ability to alleviate symptoms like pain and nausea, and for its potential anti-tumor properties, by influencing various physiological processes through the endocannabinoid system.

The term “THC”, or tetrahydrocannabinol, is the primary psychoactive compound in cannabis. In cancer, THC is studied for its potential to reduce symptoms such as pain, nausea, and loss of appetite. Additionally, research is being conducted exploring THC's potential anti-tumor effects, as THC may inhibit cancer cell growth and promote apoptosis in certain types of cancer cells through THC's interaction with the endocannabinoid system.

As used herein, the term “THCV”, or tetrahydrocannabivarin, is a minor cannabinoid found in cannabis. In the context of cancer, THCV is being studied for its potential therapeutic benefits. Research suggests that THCV may have anti-proliferative properties, meaning it could inhibit the growth of cancer cells. Additionally, THCV might possess anti-inflammatory and analgesic effects, which could help manage cancer-related symptoms and improve the quality of life for patients.

The term “CBG”, or cannabigerol, is a non-psychoactive cannabinoid found in cannabis. In cancer research, CBG is being investigated for its potential anti-tumor properties. Studies suggest that CBG may inhibit the growth of cancer cells and promote apoptosis, the programmed death of cancer cells. Additionally, CBG has shown anti-inflammatory and antioxidant properties, which could help reduce cancer-related inflammation and oxidative stress, contributing to a better overall management of the disease.

The term “CBC”, or cannabichromene, is a non-psychoactive cannabinoid found in the cannabis plant. It is being studied for its potential anti-cancer properties. CBC may inhibit cancer cell growth and promote apoptosis, the programmed death of cancer cells. Additionally, CBC has shown anti-inflammatory and analgesic properties, which help in managing cancer-related symptoms and improving patients' quality of life.

As used herein, the term “limonene” is defined as a terpene found in cannabis and other citrus fruits, known for its citrus scent. It has shown promise in cancer research due to its potential anti-cancer properties. Studies suggest that limonene may help inhibit cancer cell growth, induce apoptosis (programmed cell death), and reduce tumor size. Additionally, limonene may enhance the effectiveness of other cancer treatments by modulating various cellular pathways and reducing inflammation. Its presence in cannabis contributes to the overall therapeutic potential of cannabinoid-based treatments for cancer.

As used herein, the term “kaempferol” is a flavonoid found in many plants, including cannabis. Kaempferol is known for having antioxidant and anti-inflammatory properties. Kaempferol has shown potential in inhibiting cancer cell growth, inducing apoptosis (programmed cell death), and preventing metastasis. By modulating various signaling pathways, kaempferol can help reduce tumor growth and improve the efficacy of other cancer treatments. Its presence in cannabis enhances the therapeutic potential of cannabinoid-based treatments for cancer.

As used herein, the term “cannabinoids treatment” refers to the use of cannabinoid compounds as a therapeutic approach for pancreatic cancer. These cannabinoids derived from the cannabis plant are used for their potential anti-cancer properties, including the ability to induce apoptosis in cancer cells, inhibit tumor growth, and reduce metastasis. Additionally, cannabinoids may help alleviate symptoms associated with pancreatic cancer, such as pain, nausea, and appetite loss, thereby improving patients' quality of life. While research is ongoing, cannabinoids treatment offers a promising complementary strategy in managing pancreatic cancer.

The term “entourage effect” refers to the synergistic interaction between various compounds found in cannabis, such as cannabinoids, terpenes, and flavonoids, which enhance the overall therapeutic effects. This phenomenon shows that the combined effect of these compounds is more potent than the sum of their individual effects.

As used herein, the substances used refer to a combined therapy that includes THC, THCV, CBD, CBG, CBC, limonene, and kaempferol, each known for its potential anti-cancer properties. This comprehensive treatment aims to leverage the synergistic effects of these compounds to enhance their efficacy against pancreatic cancer. THC and CBD are well-known cannabinoids with demonstrated ability to induce cancer cell apoptosis and inhibit tumor growth. THCV and CBG contribute additional anti-inflammatory and anti-proliferative effects. CBC enhances these benefits by promoting cancer cell death, while limonene, a citrus terpene, adds antioxidant and anti-inflammatory properties. kaempferol, a flavonoid, is recognized for its ability to inhibit cancer cell proliferation and induce apoptosis. Preferably, the composition wherein THC is present in a concentration in the range of 1% to 1.45%, THCV is present in a concentration in the range of 1.5% to 2.25%, CBD is present in a concentration in the range of 4.5% to 5.5%, CBG is present in a concentration in the range of 3.5% to 4.5%, CBC is present in a concentration in the range of 1% to 1.45%, limonene is present in a concentration in the range of 25% to 28% and kaempferol is present in a concentration in the range of 25% to 28%.

As used herein, the term “treatment” refers to a “nanoemulsion capsule soft gel” containing cannabinoids designed to enhance bioavailability and absorption of the cannabinoid compounds. In an embodiment of the disclosure, the composition is suitable for oral administration in capsule form. In another embodiment, the composition is administered in an oral dosage form selected from a group consisting of capsules, tablets, or liquid solutions. The formulation may also comprise a soft gel capsule.

The term, “bioavailability” refers to the proportion of a drug or other substance that enters the circulation when introduced into the body and is able to have an active effect. Bioavailability measures how much of the administered dose reaches the systemic circulation unchanged and is available to produce a biological effect.

The term, “synergism” refers to the interaction between two or more agents, substances, or elements that produce a combined effect greater than the sum of their individual effects. In medical and pharmacological contexts, synergism often describes how different drugs or compounds work together to enhance therapeutic outcomes, improve efficacy, or reduce side effects, thereby providing a more potent and beneficial overall effect.

As used herein, the term “nanoemulsion technology” involves creating tiny, stable, droplets of cannabinoids that increase their solubility in the digestive system, ensuring more efficient and rapid uptake into the bloodstream. This improved delivery system maximizes the therapeutic potential of cannabinoids by allowing for lower dosages while achieving higher efficacy. Enhanced bioavailability ensures that a greater proportion of the active compounds reach the target sites within the body, providing more consistent and potent therapeutic effects. This approach optimizes the treatment's effectiveness for managing symptoms and combating the progression of pancreatic cancer, offering a significant advancement over traditional cannabinoid delivery methods. The term also may refer to a nanoplatform where the nanoplatform is a nanoemulsion comprising PEG-400 as a stabilizing solubilizing agent, in a form of nanoparticles and average size of less than 200 nm.

A treatment referred to “a combination therapy” is defined as alleviating symptoms for palliative care in pancreatic cancer patients. This multifaceted approach integrates various therapeutic agents and methods to manage pain, reduce nausea, improve appetite, and enhance overall quality of life. By combining medications, such as pain relievers, antiemetics, and cannabinoids, with supportive therapies like nutritional support and psychological counseling, this treatment strategy addresses the complex and multifaceted needs of palliative care patients. The goal is to provide significant relief from the debilitating symptoms associated with advanced pancreatic cancer, thereby improving comfort and maintaining dignity during the patient's remaining time.

As used herein and in the appended claims, the singular terms “a”, “an”, and “the” encompass references to the plural unless otherwise specified by the context.

It is understood that aspects and variations of the invention described herein encompass embodiments that are “limited to” and/or “substantially limited to” the described aspects and variations.

### Cannabinoids and Pancreatic Cancer

Pancreatic cancer is relatively rare but has a high mortality rate. There is a growing interest in cannabinoids, derived from the cannabis plant, as an alternative treatment for cancer patients; however, conventionally, only a limited number of studies have explored the antitumor effects of cannabinoids on pancreatic cancer.

Many cancer patients have used cannabis to alleviate symptoms caused by cancer or cancer treatments. Herbal medical plants, like cannabis, are often considered safer than conventional chemotherapy. The use of cannabis has been reported to improve the quality of life for cancer patients by reducing nausea and vomiting, stimulating appetite, and alleviating pain. Several preclinical studies and various animal cancer models have demonstrated the antitumor effects of cannabis. For instance, cannabinoids can inhibit tumor progression by inducing apoptosis and reducing tumor cell proliferation.

In pancreatic cancer, cannabinoids have been recognized for their potential antitumor effects, given that cannabinoid receptors are present in pancreatic cells and are increasingly expressed in pancreatic cancer cells. Cannabis extract has demonstrated promise as a therapeutic agent against drug-resistant cancers. Many patients with pancreatic cancer who undergo advanced chemotherapy face poor prognosis and survival rates due to the rapid development of drug resistance. As a result, cannabis extract provides a valuable alternative therapy for these patients.

Some studies have shown that cannabinoid treatment in mice can induce apoptosis and inhibit the proliferation of human pancreatic ductal adenocarcinoma cells in a dose-dependent manner. These findings suggest that cannabinoids have an antitumor effect on a human pancreatic ductal adenocarcinoma cell line (Capan-2)-derived xenograft mouse model. It is important to recognize that other compounds such as minor cannabinoids, flavonoids, and terpenes may also contribute to the observed antiproliferative effects and mediation of apoptosis. Cannabis is a remarkably intricate plant, containing over 100 different cannabinoid compounds. While CBD and THC are the major compounds that have received extensive research attention in both human patients and animal models, other cannabinoids have demonstrated therapeutic effects in various diseases, including cancer. Therefore, it is plausible that the antiapoptotic effects may be synergistically influenced by these additional cannabinoids present in cannabis extract.

One of the significant barriers to effective treatment is the immunosuppressive nature of the pancreatic tumor microenvironment, which fosters resistance to therapy. There is an urgent need for new treatment options that can enhance both the survival rates and the quality of life for patients. Current treatment options for pancreatic cancer include surgery, chemotherapy, targeted therapy, immunotherapy, and radiation therapy. Curative treatment is available only if the tumor is found early and can be removed by surgery before metastasis. If the cancer has metastasized, the standard of care is chemotherapy, or radiotherapy. Pancreatic cancer is notoriously defiant to current therapies including chemotherapy, radiotherapy, and immunotherapy.

### Natural Products a Promising Alternative Therapy for Cancer Treatment

Natural products offer a sustainable and effective source for treating a wide range of disorders, including cancer. Recently, there has been significant interest in bioactive compounds and natural products as potential sources of anti-cancer drugs, supported by collaborative, integrated, and multidisciplinary approaches. Plants have been recognized for their medicinal properties for centuries, with more than half of modern clinical drugs originating from natural sources capable of combating cancer cells.

Natural products are bioactive compounds derived from abundant natural sources like plants, known for their therapeutic properties. Many current drugs, such as alkaloids, taxanes, and flavonoids, originate from natural resources. Recently, there has been growing interest in using natural compounds in chemotherapy to enhance the effectiveness of anti-cancer drugs. These compounds, including phytochemicals, minerals, and vitamins, show promising results in treating various malignant tumors, often with minimal toxicity to healthy tissues. Research suggests combining natural products with traditional anti-cancer drugs to explore synergistic effects. Natural compounds exhibit selective benefits against cancer cells while serving as models for developing novel drugs with improved efficacy and reduced side effects and resistance.

Chemoenzymatic biotransformation, using natural products as substrates to generate diverse derivatives, is a promising approach. This method, leveraging the catalytic diversity of fungal secretomes, has successfully produced libraries of stilbene dimers and phenylpropanoids. Phenylpropanoids, like caffeic and ferulic acids, known for their antioxidant properties, serve as crucial building blocks in drug discovery. Previous studies have demonstrated the transformation of these compounds into more complex derivatives using purified enzymes, highlighting their potential as effective chemotherapeutic agents due to their selectivity, cost-effectiveness, and low toxicity.

Although various anti-cancer drugs are available, their toxic effects often limit their use. Given the severe side effects of radiotherapy and chemotherapy and their high mortality rates, current research is increasingly focused on designing effective cancer treatments with minimal side effects. Biodiversity remains a critical source of potent anti-cancer agents to date cells.

### Pharmacological Profiles of Cannabinoids

In embodiments of the disclosure, phytocannabinoids interact with the endocannabinoid system (ECS) in humans and animals, crucial for regulating functions like mood, appetite, and pain perception. By binding to ECS cannabinoid receptors (CB1 and CB2), these compounds influence physiological processes, enhancing their therapeutic potential. It has been found, in example embodiments of the disclosure, that cannabis exhibits an “entourage effect,” where terpenes and phytocannabinoids together produce effects greater than their individual contributions. These synergistic effects are found in a number of substances listed below. This synergy emphasizes the importance of using the whole plant in medicinal applications, maximizing therapeutic benefits through complex interactions between its compounds.

### Δ9-Tetrahydrocannabinol (THC)

THC is the primary psychoactive compound in cannabis, characterized by its complex, bicyclic structure. It's absorbed well through inhalation or ingestion, accumulating in fatty tissues and metabolized by hepatic enzymes. THC alters perception and mood, causing the characteristic “high.” It is used medically for pain relief and antiemetic effects. Legal classifications vary due to its psychoactive nature.

### Cannabidiol (CBD)

CBD, a non-psychoactive compound, has a tricyclic structure and minimal affinity for CB1 receptors, avoiding psychoactive effects. It interacts with CB2 receptors, influencing inflammation and immune responses. CBD is known for its anti-inflammatory, analgesic, anxiolytic, and neuroprotective properties. Epidiolex® is approved for epilepsy treatment. Research on its anti-cancer potential is ongoing.

### Cannabigerol (CBG)

CBG has a complex structure with lower affinity for CB1 and CB2 receptors. It acts as a partial agonist or antagonist at CB1 receptors, influencing other cannabinoids' effects. CBG shows neuroprotective, anti-inflammatory properties, and stimulates appetite, with potential benefits in inflammatory bowel disease and cancer research.

### Other Phytocannabinoids

### Cannabichromene (CBC)

CBC interacts with the ECS but has low affinity for CB1 and CB2 receptors. It's recognized for anti-inflammatory and potential neuroprotective effects, contributing to pain relief and CNS disorder treatments.

### Cannabinol (CBN)

CBN results from THC degradation, with mild psychoactive effects via CB1 receptor partial agonism. It exhibits sedative properties and enhances cannabis-induced sedation and pain relief.

### Tetrahydrocannabivarin (THCV)

THCV interacts with the ECS, showing higher affinity for CB2 receptors. THCV effects range from CB1 receptor antagonism at low doses to partial agonism at higher dose.

Cannabinoids represent a diverse group of compounds increasingly explored for their therapeutic potential across various medical conditions. While research often emphasizes efficacy; comprehensive safety data remains limited and overshadowed by associations with recreational cannabis use. Factors such as study design variations, indications, dosing, and administration methods further complicate safety assessments. In aspects of the disclosure presented below, these factors are addressed to provide a safe and effective treatment of pancreatic cancer and its symptoms.

Among cannabinoids, Δ9-tetrahydrocannabinol (THC) and cannabidiol (CBD) have been extensively studied. Common adverse events with THC or THC-CBD combinations include dizziness and fatigue. THC usage may also lead to cognitive impairment and affect psychomotor skills, potentially impacting activities like driving. CBD, conversely, may mitigate THC's psychotropic effects when co-administered. Studies on dependency and addiction in medical settings show mixed results, underscoring the need for further investigation. Such factors of dependency and addiction are addressed in embodiments of the disclosure, solving a long felt need in the medical field.

It has been found, in example embodiments, that oral ingestion is typically recommended over smoking due to concerns about potential release of mutagenic and carcinogenic by-products. Upon such oral ingestion treatments as described, physicians should consider potential drug interactions and contraindications when prescribing cannabinoid-based medicines.

As found herein, a synergy between THC and CBD is recognized. When used together, the synergy complements each other's therapeutic effects while mitigating adverse reactions like cognitive impairment associated with THC alone. It has been found that this combination, previously unknown, opens new avenues for personalized medicine approaches tailored to individual patient needs and conditions. As such, treatments presented herein can be tailored to individual patients to maximize the beneficial effects of the synergistic effects discovered.

In the realm of medical research, the synergistic effects described above address unmet medical needs and improve quality of life for patients, contrary to conventional medicines and treatment technologies. A precise application of compounds; however, is needed to maximize beneficial effects. Administration of doses outside the identified ranges decreases or eliminates the beneficial effects of the compounds and any synergistic effects that are present.

### Cannabinoids Treatment for Pancreatic Cancer

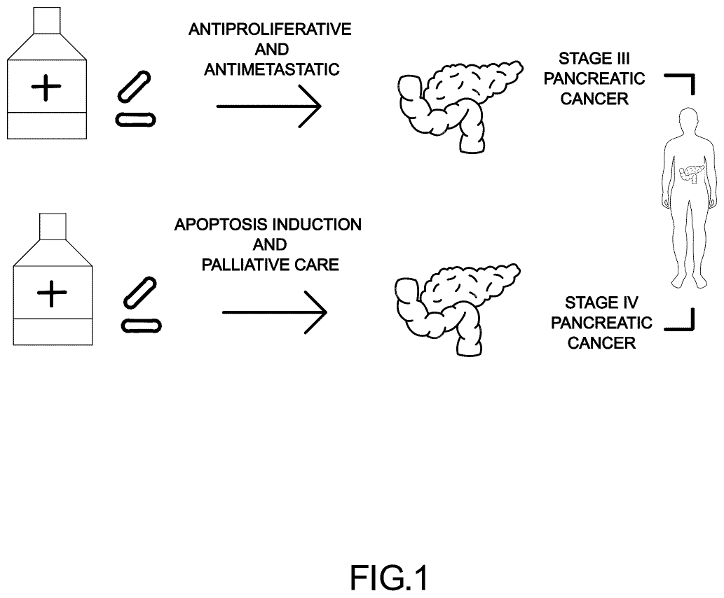

FIG. 1. illustrates an overview of aspects of the disclosure relating to patients with stage III and IV pancreatic cancer. It highlights enhanced and broadened palliative care by suppressing tumor growth, reducing metastatic activity, and mitigating the aggressiveness of the disease. Additionally, the aspects shown act as an adjuvant therapy in the clinical management of symptoms such as pain relief, nausea, and cachexia associated with pancreatic cancer. In FIG. 1, a patient, at the right of the FIG., can be treated for either stage Ill pancreatic cancer (top portion of FIG.) or stage IV pancreatic cancer (bottom portion of FIG.)

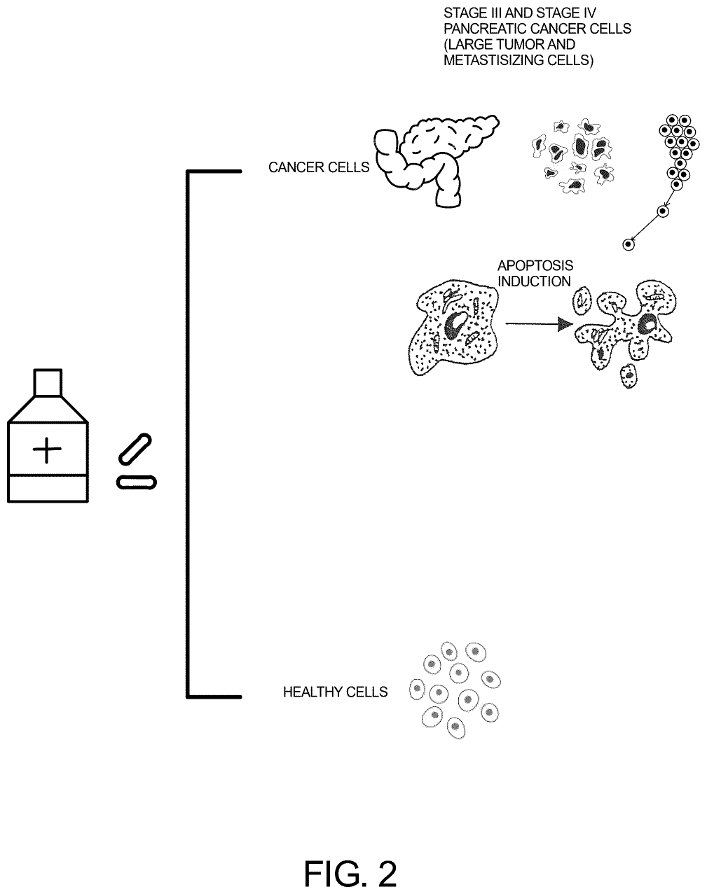

FIG. 2. illustrates the effects and mechanism of action of aspects of the disclosure in pancreatic cancer cells, as well as its role in protecting and enhancing cellular response in healthy cells. The aspects described exhibit antiproliferative, antimetastatic, and antiangiogenic properties, indicating its ability to inhibit the growth of cancer cells, prevent metastasis, and reduce the formation of new blood vessels crucial for tumor growth. In FIG. 2, left, the mechanism of action for healthy cells (bottom of FIG.) is to prevent or intervene in tumor cell development cycles, inhibit growth, induce apoptosis and reduce tumor cell migration and angiogenic activity. For cancer cells (top of FIG.), cancer is inhibited from proliferation, including apoptosis and metastasis, modulating signaling pathways, reducing inflammation and interfering with cancer cell migration and invasion.

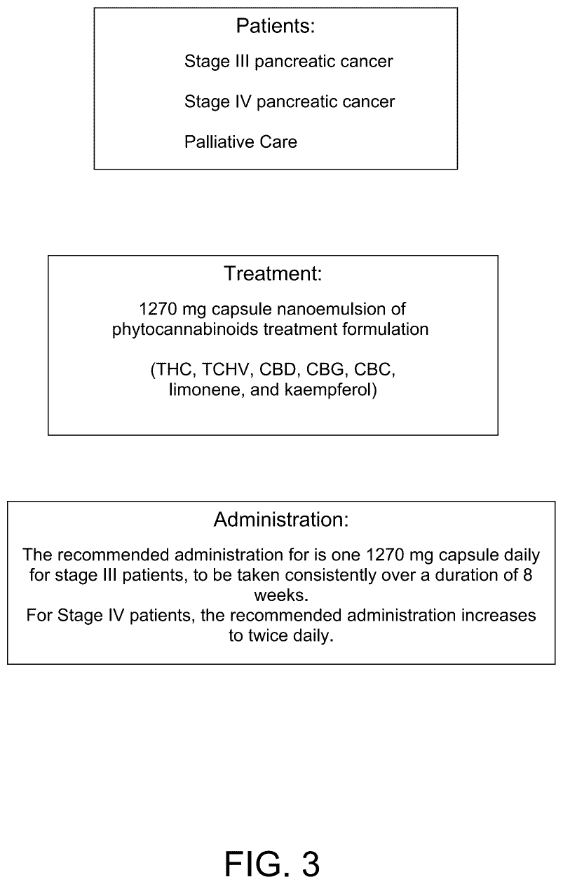

FIG. 3. illustrates the treatment approach of aspects of the disclosure focusing on oral administration of a comprehensive formulation containing cannabinoids, terpenes, and flavonoids. This treatment strategy targets patients with stage III and IV pancreatic cancer as well as those in palliative care, aiming to delay cancer progression and enhance patients' quality of life.

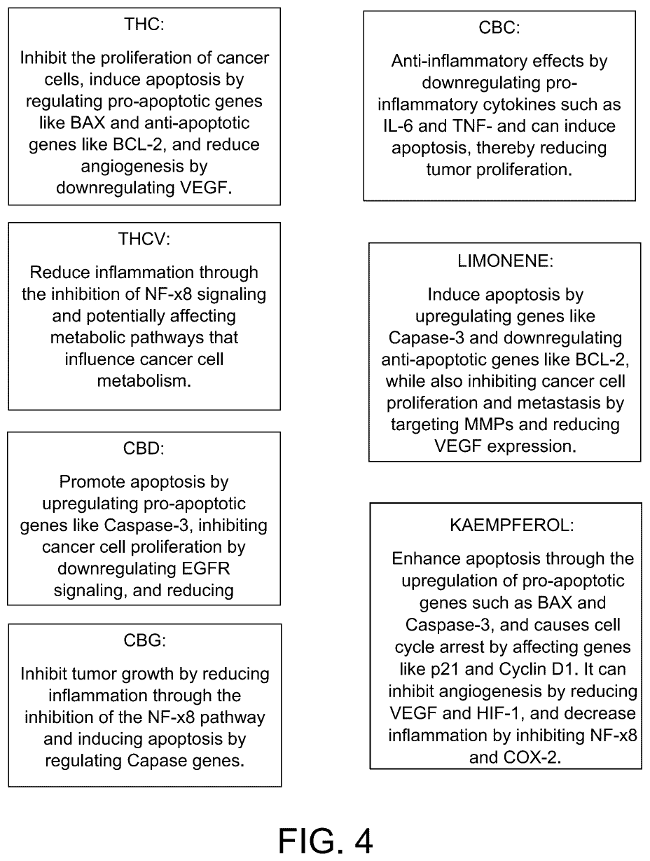

FIG. 4. illustrates the anti-cancer properties of cannabinoids THC, THCV, CBD, CBG, and CBC, the terpene limonene, and the flavonoid kaempferol. In the embodiments illustrated, these compounds target specific genes involved in cancer progression, wherein aspects of the disclosure provide a treatment designed for stage III and IV pancreatic cancer. In the embodiments shown, the active compounds work synergistically to induce apoptosis, inhibit tumor growth and metastasis, reduce inflammation, and impede angiogenesis, offering a comprehensive therapeutic approach for advanced pancreatic cancer.

Cannabinoids, which are the bioactive components of Cannabis sativa L. and their derivatives, have been investigated as both anti-cancer agents and for managing the side effects of conventional cancer treatments like chemotherapy and radiotherapy. Previous studies have indicated that medical cannabis derivatives could enhance survival in pancreatic cancer animal models, when used in synergy with radiotherapy. While general research has been conducted, studies of precise application of amounts of cannabinoids have not occurred.

PDAC is resistant to most available chemotherapy and radiation treatments, making surgery the most effective curative option. The desmoplastic tumor microenvironment plays a crucial role in PDAC pathophysiology, immune response, and therapeutic efficacy.

Current therapeutic approaches for PDAC, including FDA-approved chemotherapeutics like gemcitabine, braxane, and folfirinox, only marginally prolong survival and are associated with adverse effects. Thus, there is a long felt need for treatment options for PDAC. Pharmacological targeting of the endocannabinoid system offers therapeutic benefits for pancreatic cancer as found herein, providing an alternative to conventional medicines and methodologies.

Phytocannabinoids, the active compounds found in cannabis, have shown potential in the treatment of pancreatic cancer. They display anti-cancer effects in several models by suppressing the proliferation, migration, and/or invasion of cancer cells, as well as tumor angiogenesis. Cannabinoid receptors have been shown to be much more highly expressed than in regular tissues. Treatment of pancreatic cancer cells with THC was able to decrease cell viability, induce apoptosis, and reduce the growth of tumors in vivo in murine models. The induction of apoptosis by THC in pancreatic cancer cells was the result of ceramide accumulation and endoplasmic reticulum stress, as shown by increased expression of stress protein p8 mRNA.

Research indicates that phytocannabinoids, like CBD and THC, can bind to CB2 receptors, leading to several anti-cancer effects. These effects include inhibiting cancer cell proliferation, inducing apoptosis, reducing tumor growth, and diminishing metastasis. Additionally, CB2 receptor activation can modulate immune responses and decrease inflammation, further contributing to the potential therapeutic benefits of phytocannabinoids in treating pancreatic cancer.

CB1 and CB2 receptors are present in both exocrine and endocrine pancreatic tissues, suggesting that the endocannabinoid system (ECS) plays a significant role in regulating pancreatic secretion. In human islets of Langerhans, CB1 receptors are densely located in glucagon-secreting alpha cells and less so in insulin-secreting beta cells. CB2 receptors are largely expressed in somatostatin-secreting delta cells but are absent in alpha and beta cells. In rats, CB1 and CB2 receptors are expressed in pancreatic lobules, with CB1 showing higher expression than CB2, as well as in pancreatic acini. It has been shown that CB1 and CB2 receptors are overexpressed in human pancreatic tumor cell lines and biopsies. Cannabinoids selectively reduce pancreatic cancer cell growth both in vitro and in vivo, compared to non-transformed pancreatic cells.

### Anticancer Properties of Cannabinoids in Pancreatic Cancer

Anti-tumor Effects: Some studies suggest that cannabinoids can inhibit the growth of pancreatic cancer cells. They can induce apoptosis and autophagy in cancer cells, which helps to reduce tumor size.

Anti-inflammatory Properties: Cannabinoids have anti-inflammatory effects, which can be beneficial in reducing the inflammation associated with pancreatic cancer. Chronic inflammation is known to contribute to cancer progression, so reducing it can help manage the disease.

Anti-metastatic Properties: Some research indicates that cannabinoids can inhibit the metastasis of cancer cells to other parts of the body. This is particularly important in pancreatic cancer, which is often diagnosed at an advanced stage with metastasis.

Appetite Stimulation: Cachexia and loss of appetite are common in pancreatic cancer patients. Cannabinoids are known to stimulate appetite, which can help maintain nutritional intake and improve the quality of life.

Chemotherapy Synergy: There is evidence that cannabinoids can enhance the efficacy of certain chemotherapy drugs. They may help to sensitize cancer cells to chemotherapy, making the treatment more effective.

Nausea and Vomiting Control: Cannabinoids are effective in controlling nausea and vomiting, which are common side effects of chemotherapy. This can help improve patient comfort and adherence to treatment.

Pain Relief: Pain management is a crucial aspect of pancreatic cancer care. Cannabinoids are known for their analgesic properties, which can help alleviate the severe pain experienced by patients.

### Anticancer Properties of Phytocannabinoids

Phytocannabinoids such as CBD, CBC, THC, THCV, CBN, CBG, and others show promising potential as anti-cancer agents, particularly in pancreatic cancer treatment.

CBD: Inhibits cancer cell growth and induces apoptosis. Reduces inflammation and angiogenesis crucial for tumor growth.

CBC: Inhibits tumor growth and cancer cell proliferation. Interacts with the endocannabinoid system to modulate cancer pathways.

THC: Exhibits cytotoxic effects on cancer cells and induces apoptosis. Reduces pain and inflammation associated with cancer.

THCV: Inhibits cancer cell proliferation and enhances chemotherapy effectiveness.

CBG: Inhibits cancer cell growth and induces apoptosis in various cancer types.

### Cannabis Terpenes and Flavonoids Importance in Pancreatic Cancer Treatment

Limonene, a terpene found in cannabis, is a therapeutic agent against pancreatic cancer. Research has shown that limonene inhibits the proliferation of pancreatic cancer cells. This in turn slows tumor growth. Limonene also induces apoptosis. Furthermore, limonene's anti-inflammatory properties help reduce inflammation associated with pancreatic cancer, thereby contributing to therapeutic effects. As an antioxidant, limonene protects cells from oxidative damage, which is implicated in cancer development. Limonene's ability to work synergistically with other compounds in cannabis suggests enhanced efficacy in combating pancreatic cancer.

Flavonoids found in cannabis, such as kaempferol, also help in the treatment of pancreatic cancer. Kaempferol is recognized for antioxidant properties, which help protect cells from oxidative damage, a significant factor in cancer development. Additionally, flavonoid's anti-inflammatory effects reduce inflammation in pancreatic tissues, which often contributes to cancer progression. Studies have indicated that kaempferol can inhibit the growth of pancreatic cancer cells and induce apoptosis, thereby hindering tumor advancement. Furthermore, kaempferol's ability to modulate key signaling pathways involved in cell growth and survival suggests flavonoids may disrupt mechanisms that promote pancreatic cancer. Flavonoids' potential to inhibit angiogenesis further underscores its role in impeding tumor growth and metastasis.

### Nanoemulsion of Phytocannabinoids in Drug Delivery of Pancreatic Cancer Treatment

The discovery of CB1 and CB2 endogenous cannabinoid receptors has sparked extensive research into the endocannabinoid system. These receptors are also important in regulatory functions in health and disease. Medicinal cannabis, particularly cannabinoids like tetrahydrocannabinol and cannabidiol, show significant therapeutic potential. These compounds have demonstrated efficacy in managing chronic pain, reducing spasticity and inflammation in neurodegenerative disorders, and alleviating chemotherapy-induced nausea and vomiting. An embodiment of the disclosure refers to a method for producing the pharmaceutical composition for treating pancreatic cancer, comprising: Incorporating a cannabinoid, a terpene, and a flavonoid into a nanoplatform comprising a stabilizing solubilizing agent.

It has been found that nanoemulsion formulations of cannabinoids and natural compounds offer several advantages in drug delivery. They enhance the solubility and bioavailability of these compounds, leading to improved absorption and therapeutic effects.

Nanoemulsions provide better stability and can be administered through various routes, including oral, intravenous, rectal, topical, and nasal, offering versatility in treatment options. In embodiments of the disclosure, cannabis nanoemulsions are emulsions characterized by small droplet size, offering numerous advantageous properties for cannabinoid formulations. These include enhanced clarity, stability, and low viscosity.

In embodiments of the disclosure, a typical nanoemulsion concentrate for pharmaceutical drug delivery contains an oil phase, consisting of the active lipophilic compound dissolved in a carrier oil, surfactants, and water. Medium-Chain Triglycerides (MCT) carrier oils are commonly used in nanoemulsion formulations as they simplify processing by lowering the viscosity of the oil phase and as mentioned above, can increase the bioavailability of lipophilic drugs.

Surfactants are amphiphilic species with polar heads and nonpolar tails that preferentially adsorb to water/oil interfaces and are essential to nanoemulsion formation because they reduce the surface tension between the two immiscible phases and, therefore, lower the energy barrier that has to be overcome during the nanoemulsion formation. Surfactants are also critical to the long-term stability of nanoemulsions as they sustain small droplets, thereby allowing Brownian motion to overwhelm any gravitational separation routes.

Oral administration is generally preferred for pharmaceuticals due to improved patient compliance. However, cannabinoids' lipophilic nature and low aqueous solubility leads to challenges such as low bioavailability (<20 percent) and delayed peak concentrations (>2 h) when administered orally. These factors present substantial hurdles in designing effective cannabinoid formulations for systemic delivery in therapeutic applications.

Given the difficulties with oral delivery, alternative administration routes for cannabinoids are more commonly employed. The oldest method is pulmonary delivery via smoking, which involves inhaling smoke from burning cannabis plant matter. Smoking bypasses first-pass metabolism, allowing for rapid onset of effects. However, smoking is not ideal for medicinal use due to the harmful carcinogenic by-products produced during combustion, which can damage the lungs. Moreover, many patients, particularly the elderly, may find smoking harsh and be non-compliant.

As an alternative to smoking, vaporization (vaping) of cannabis plant matter or extracted oils has gained popularity. Vaping is sometimes touted as a safer option compared to smoking, although these claims lack substantial evidence. Similar to nicotine-based vaping, it should not be considered harmless. Additionally, vaping suffers from variability in inhalation patterns among patients, leading to unpredictable drug exposure and varying bioavailability rates (2 percent-50 percent).

While cannabinoids like THC and CBD offer therapeutic promise, the challenge lies in optimizing their delivery methods to enhance efficacy and minimize risks associated with administration routes such as smoking and vaping.

Nanoemulsions are advanced drug delivery systems that enhance the bioavailability and efficacy of phytocannabinoids. These nano-sized emulsions improve the solubility of phytocannabinoids like cannabidiol and tetrahydrocannabinol, which are naturally hydrophobic and have limited absorption in the body. By encapsulating phytocannabinoids nanoemulsions, their stability is increased, and their release can be controlled, leading to more effective and sustained therapeutic effects. Embodiments of the disclosure provide these delivery systems, previously not offered in cannabis administration, to allow for enhanced effects that have been discovered herein. Preferably, the nanoemulsion formulation is designed to provide a controlled release of cannabinoids, terpenes, and flavonoids.

In the context of pancreatic cancer treatment, nanoemulsions, such as used in example embodiments, offer several benefits. Nanoemulsions facilitate targeted delivery of phytocannabinoids to cancer cells, potentially enhancing the anti-cancer effects by improving cellular uptake and distribution. Additionally, the small size of nanoemulsions allows for better penetration into tumor tissues, overcoming some of the barriers associated with traditional drug delivery methods. This targeted approach not only maximizes the therapeutic potential of phytocannabinoids but also minimizes potential side effects, making nanoemulsions as used herein, effective against pancreatic cancer.

### EXAMPLES

Provided herein are methods for treating pancreatic cancer in individuals using a composition consisting of nanoparticles based on the nanoemulsion technique, incorporating phytocannabinoids, terpenes, and specifically targeting stage III, IV, and palliative care patients. Aspects of the disclosure function through a multifaceted mechanism to support pancreatic health and combat pancreatic cancer. These aspects target cancer cells by inducing apoptosis and inhibiting proliferation, thereby reducing tumor growth. The formulation includes active phytocannabinoid compounds that modulate key signaling pathways involved in cancer progression, such as the PI3K/Akt and MAPK pathways. This formulation targets key genes involved in pancreatic cancer, including KRAS, TP53, CDKN2A, SMAD4, EGFR, and VEGF. An embodiment of the disclosure comprises a method for treating metastatic or locally advanced pancreatic cancer in a human individual, comprising: administering an effective concentration of the composition of the disclosure, wherein the composition comprising a) an active principle and b) stabilizing solubilizing agent, wherein the active principle comprising a cannabinoid, a terpene, and a flavonoid.

Aspects disclosed above exhibit anti-inflammatory properties, reducing the chronic inflammation often associated with pancreatic cancer solving a long felt need in treatment options for pancreatic cancer. These aspects also enhance the immune system's ability to recognize and attack cancer cells while protecting normal pancreatic cells from damage. Through these combined actions, aspects of the disclosure provide a comprehensive approach to managing pancreatic cancer and improving overall pancreatic function.

An embodiment of the disclosure correspond to a composition for the treatment of pancreatic cancer, comprising a) an active principle and b) stabilizing solubilizing agent, wherein the active principle comprising a cannabinoid, a terpene, and a flavonoid.

In one non-limiting embodiment of the disclosure, the composition of the disclosure, wherein the active principle is present in a concentration in the range of 50% to 71%, and the b) stabilizing solubilizing agent is present in a concentration in the range of 0.1% to 5%. In a further embodiment, the composition of the disclosure wherein the cannabinoid is present in a concentration in the range of 10% to 15%, terpene is present in a concentration in the range of 25% to 28%, and flavonoid is present in a concentration in the range of 25% to 28%. Also, the formulation is administered orally in capsule form, with each capsule containing a concentration of 1270 mg of the principle active. This treatment will be taken daily for a duration of 8 weeks. The capsules are developed to use a nanoemulsion technique to enhance the bioavailability and efficacy of the active compounds. It has been found that using a nanoemulsion technique is superior to other administration techniques, thus providing a better outcome for patients. Such administration allows for quick and convenient applications without the drawbacks of other administration techniques.

In an embodiment of the disclosure, in the composition THC is present in a concentration in the range of 1% to 1.45%, THCV is present in a concentration in the range of 1.5% to 2.25%, CBD is present in a concentration in the range of 4.5% to 5.5%, CBG is present in a concentration in the range of 3.5% to 4.5%, CBC is present in a concentration in the range of 1% to 1.45%, limonene is present in a concentration in the range of 25% to 28% and kaempferol is present in a concentration in the range of 25% to 28%. It has been found that the recommended administration for aspects of the disclosure is one 1270 mg of the principle active, capsule daily for stage Ill patients, to be taken consistently over a duration of 8 weeks. For stage IV patients, the recommended administration increases to twice daily. It has been found that this regimen optimizes the therapeutic effects of cannabinoids and bioactive compounds contained in the capsule, supporting symptom management and potentially enhancing overall quality of life during treatment.

Aspects of the disclosure may be tailored for patients in advanced stages of pancreatic cancer, specifically stage III and IV. These aspects aim to achieve several critical objectives: first, by targeting key molecular pathways, the administered dose delays tumor invasiveness, thereby slowing down disease progression and potentially extending patient survival. Second, the administered dose aims to prevent metastasis, reducing the spread of cancer cells to other parts of the body and lowering associated health risks. Furthermore, this formulation enhances the tumor microenvironment to be less conducive to tumor growth and supportive of other therapeutic interventions. By modulating these conditions, the aspects described optimize the effectiveness of additional treatments. Preferably, method of the disclosure, wherein the human individual is selected for treatment based on at least one of the following: i) Stage III pancreatic cancer, ii) Stage IV metastatic pancreatic cancer, iii) Pancreatic cancer in the pancreatic head, iv) More than three metastatic sites, v) Patients under palliative care.

Additionally, it has been found that aspects of the disclosure inhibit tumor angiogenesis, disrupting the blood supply to tumor cells and depriving the tumor cells of vital nutrients and oxygen needed for growth and survival. Together, these mechanisms improve patient outcomes, alleviate symptoms, and enhance overall quality of life for individuals battling advanced pancreatic cancer.

The decision to administer the formulations presented herein once daily for stage III and twice daily for stage IV pancreatic cancer patients is grounded in several clinical considerations. It has been found that stage IV pancreatic cancer typically signifies a more advanced disease state with heightened symptom severity, necessitating a more frequent dosing regimen to effectively manage symptoms such as pain, nausea, and appetite loss. Increasing the frequency of administration to twice daily ensures a consistent presence of cannabinoids and bioactive compounds in the bloodstream, optimizing their therapeutic effects throughout the day. As will be understood, bioavailability may vary throughout the day, complicating treatment. Conventional approaches to treatment do not address such complications. Aspects of the disclosure herein identify the varying bioavailability and metabolic responses among patients and maintain adequate levels of active ingredients for enhanced therapeutic outcomes. By tailoring the dosing schedule to the evaluated disease stage, healthcare providers can better address the evolving needs of patients, supporting symptom control and overall quality of life during treatment.

Embodiments of the disclosure may also be used in palliative care in patients with pancreatic cancer. This innovative treatment combines cannabinoids, such as THC, THCV, CBD, CBG, CBC, with terpenes, and flavonoids, all encapsulated using a nanoemulsion technique to enhance bioavailability and therapeutic efficacy. In one non-limiting embodiment, this formulation for palliative care contains the following active ingredients per dosage: THC-25 mg; THCV-40 mg; CBD-100 mg; CBG-80 mg; CBC-25 mg; limonene-500 mg; and kaempferol-500 mg.

Aspects of the treatment described above offer numerous benefits for palliative care patients with advanced pancreatic cancer that are unavailable with conventional treatment formulations and techniques. The aspects disclosed provide significant pain relief and effectively manage symptoms like nausea, appetite loss, and fatigue, thereby enhancing overall well-being and improving quality of life. The formulation's anti-inflammatory properties help mitigate inflammation and related complications in patients. Moreover, the aspects described complement conventional therapies such as chemotherapy and radiotherapy by reducing their side effects and boosting efficacy through synergistic effects. Thus, aspects of the disclosure may be used in supplementing conventional technologies providing an enhancement in patient care previously unobtainable without the formulations described.

Aspects of the disclosure offer a multifaceted approach to the treatment of advanced pancreatic cancer, addressing both the direct and indirect factors contributing to disease progression and patient discomfort. Moreover, the combination is particularly effective in pancreatic cancer patients with a poor prognosis. This includes, but is not limited to, patients with late-stage pancreatic cancer, those with three or more metastatic sites, patients with metastases in the liver or lungs, or other parts of the body, and individuals with a serum CA19-9 level of 59 times the upper limit of normal (ULN). Additionally, the treatment shows promise for patients with a poor performance status, such as those with a Karnofsky Performance Status (KPS) score of less than 90, particularly within the 70-80 range. It is also notably effective for patients whose primary pancreatic cancer is located in the head of the pancreas.

The formulation for palliative care containing the active ingredients per dosage of THC-25 mg, THCV-40 mg, CBD-100 mg, CBG-80 mg, CBC-25 mg, limonene-500 mg and kaempferol-500 mg, are carefully selected to provide therapeutic benefits in the treatment of stage III and IV pancreatic cancer. It has been found that formulations outside of these dosages are not as beneficial to patients.

Each component of the formulation plays a specific role in addressing pancreatic cancer through various mechanisms as follows:
THC: exhibits potential anti-tumor properties by binding to cannabinoid receptors (CB1 and CB2) in cancer cells. This interaction may induce apoptosis and inhibit cancer cell proliferation.
THCV: Interacts with cannabinoid receptors, potentially influencing appetite and metabolism. It involves modulating signaling pathways involved in cancer progression.
CBD: Anti-inflammatory properties, CBD interacts with various receptors and ion channels in the body. It may inhibit tumor growth, induce apoptosis, and reduce inflammation in pancreatic cancer cells.
CBG: Anti-inflammatory and neuroprotective effects. It may also inhibit the growth of cancer cells and contribute to reducing inflammation associated with pancreatic cancer.
CBC: Anti-inflammatory and analgesic properties. CBC may inhibit the uptake of anandamide, a neurotransmitter involved in pain perception and inflammation.
Limonene: A terpene that contributes to the entourage effect by enhancing the absorption of cannabinoids and imparting its own anti-cancer effects. In correct dosages limonene inhibits tumor growth and metastasis through mechanisms that include apoptosis induction and antioxidant activity.
Kaempferol: A flavonoid with antioxidant and anti-inflammatory properties, kaempferol plays a role in reducing oxidative stress and inflammation associated with pancreatic cancer. It also contributes to the overall antioxidant defense system in the body.

The entourage effect in the context of treatment for stage III and IV pancreatic cancer refers to the synergistic interaction of multiple cannabinoids and other active compounds found in the formulation.

The entourage effect enhances therapeutic effects. Cannabinoids like THC, CBD, CBG, CBC, and THCV, along with terpenes, and flavonoids, work together synergistically. This synergy enhances their individual therapeutic properties, increasing efficacy in combating pancreatic cancer cells.

The entourage effect also contributes to better symptom management. Cannabinoids have shown anti-inflammatory properties that can help reduce pain and inflammation associated with pancreatic cancer. Terpenes like limonene can enhance mood and reduce nausea, common symptoms in cancer patients undergoing treatment.

Different cannabinoids interact with cannabinoid receptors (CB1 and CB2) in the endocannabinoid system, influencing various physiological processes including immune response and cell proliferation. This modulation can affect tumor growth and progression in pancreatic cancer.

By harnessing the entourage effect, aspects of the lower the required dosage of individual cannabinoids and mitigate side effects associated with high doses of isolated compounds.

It has been found through experimentation that cannabinoids and natural products are often considered safer than conventional drugs for several reasons, making them a promising alternative or complement to chemotherapy and radiotherapy in cancer treatment. One key factor is their natural origin, which often results in higher biocompatibility with the human body. Cannabinoids like THC, CBD, CBG, and CBC, along with natural compounds such as limonene and kaempferol, generally exhibit lower toxicity profiles compared to synthetic chemotherapy drugs. This means they typically cause fewer and less severe side effects, such as nausea, fatigue, and immunosuppression.

These natural agents, and the treatment described herein utilizing such agents, offer specific targeting capabilities. Cannabinoids and natural compounds can target specific pathways involved in cancer cell growth and survival, sparing healthy cells and reducing the risk of off-target effects. For example, the cannabinoids and natural compounds can induce apoptosis in cancer cells selectively, which minimizes collateral damage to normal tissues. This selective action contrasts with conventional chemotherapy and radiotherapy, which can indiscriminately harm both cancerous and healthy cells. Thus, treatment options utilizing the formulations described as well as methods presented are less harmful to the use of conventional chemotherapy.

The aspects disclosed using the formulations provided use a multi-targeted approach that can be particularly beneficial in treating complex diseases like pancreatic cancer. The formulations presented act on multiple biological pathways, addressing various aspects of cancer progression, including inflammation, cell proliferation, and metastasis. This synergies discovered allow for the possibility of using lower doses of conventional chemotherapy or radiotherapy, thereby reducing the associated toxicities and improving patient tolerability. The reduction of such conventional chemotherapy and radiotherapy treatments also decreases the overall economic cost of treatment for patients. Conventional therapies may be economically prohibitive for some patients without extensive financial means, thereby limiting treatment options. By providing options that treat the disease and its associated symptoms and limiting the amount or cost of conventional therapies, overall economics for treatment are achieved that are not found in conventional technologies.—The long felt need for providing an economical treatment option for pancreatic cancer is therefore achieved with aspects of the disclosure compared to the more limiting options available in conventional technologies.

Another significant advantage is the improved patient tolerability associated with cannabinoids and natural products. Patients undergoing treatment with these agents often experience fewer and less severe side effects compared to those receiving traditional chemotherapy or radiotherapy. Additionally, cannabinoids are effective in managing symptoms such as pain, nausea, and appetite loss, which are common in cancer patients. This symptom management further improves the overall quality of life for patients during treatment. Such advantages are not present in conventional technologies and represent a significant breakthrough in treatment.

Aspects of the disclosure enhance conventional therapies. Such applications promote advantages that cannot be achieved by conventional treatments alone. The method and formulations described can sensitize cancer cells to these treatments, reducing resistance, and potentially allowing for lower doses of conventional drugs. This complementary action not only boosts treatment effectiveness but also helps in reducing the severity of side effects associated with high-dose chemotherapy and radiotherapy. Overall, cannabinoids offer a comprehensive approach to cancer treatment, supporting both the direct anti-cancer effects and the overall well-being of the patient not achieved with conventional technologies, methods and formulations.

### Methods of Treatment Pancreatic Cancer

Example embodiments of the disclosure may be altered in a range by plus or minus ten percent as described in this specification. In some embodiments, the nanoemulsion formulation for embodiments of the disclosure combine a precise blend of cannabinoids and bioactive compounds aimed at therapeutic efficacy. In some embodiments, each 1270 mg of the principle active, in a capsule, includes 25 mg of THC, 40 mg of THCV, 100 mg of CBD, 80 mg of CBG, and 25 mg of CBC, known for their potential anti-inflammatory, analgesic, and anti-cancer properties. In some embodiments, the formulation includes 500 mg of limonene and 500 mg of kaempferol, enhancing the entourage effect to potentially improve mood, reduce nausea, and provide antioxidant benefits. In some embodiments, this nanoemulsion technology ensures optimal absorption and bioavailability of these compounds, potentially offering enhanced therapeutic benefits for conditions such as pancreatic cancer or pancreatitis.

In some embodiments, administration occurs once daily over an 8-week period for treating stage III pancreatic cancer. In some embodiments, this treatment regimen leverages cannabinoids and bioactive compounds known for their targeted effects on gene pathways involved in cancer progression. In some embodiments, these compounds interact with cannabinoid receptors and other molecular targets to influence apoptosis, inflammation, and cellular signaling pathways in managing tumor growth and metastasis.

In some embodiments, for stage IV pancreatic cancer, administering embodiments of the formulations described, twice daily over the same duration is suitable. In some embodiments, this increased frequency is designed to optimize therapeutic levels of these compounds throughout the day, addressing more advanced disease stages and potentially enhancing treatment efficacy by managing symptoms and influencing disease progression. In some embodiments, this regimen allows for consistent administration of the cannabinoids and bioactive compounds, potentially providing sustained benefits in managing symptoms and supporting overall health during the treatment period.

In some embodiments, the duration of 8 weeks aligns with typical treatment schedules aimed at assessing initial response and evaluating the tolerability of the therapy. It also allows sufficient time for the body to potentially respond to the therapeutic effects of the formulation.

In some embodiments, the optimal dosing regimen may vary based on individual patient factors, including the severity of symptoms, overall health status, and response to treatment.

In some embodiments, medical supervision and regular monitoring are conducted during this period to assess efficacy, manage any potential side effects, and adjust the treatment plan as needed. This approach aims to maximize the therapeutic benefits of the formulations described while ensuring patient safety and comfort throughout the treatment course.

In some embodiments, cannabinoids such as THC, THCV, CBD, CBG, CBC, along with bioactive compounds like limonene and kaempferol, interact with various molecular pathways implicated in pancreatic cancer treatment. In some embodiments, THC and CBD primarily engage cannabinoid receptors CB1 and CB2, influencing pathways related to cell survival, apoptosis, and inflammation. In some embodiments, CBD additionally modulates the NF-κB pathway, impacting inflammation and immune responses crucial in cancer progression. In some embodiments, CBG and CBD affect the PI3K/Akt/mTOR pathway, which regulates cell growth and proliferation. CBD also interacts with the p53 pathway, involved in cell cycle control and apoptosis. In some embodiments, Limonene and kaempferol influence the MAPK pathway, crucial for cell survival and proliferation signaling. In some embodiments, these interactions suggest a complex interplay where cannabinoids and bioactive compounds potentially contribute to managing symptoms and influencing the progression of pancreatic cancer through diverse molecular mechanisms.

In some embodiments, creating a cannabis nanoemulsion formulation, as described, involves several key steps to ensure stability, bioavailability, and therapeutic efficacy. In some embodiments, the formulation development determines the specific composition of cannabinoids, terpenes, and other bioactive compounds based on their desired therapeutic effects for pancreatic conditions.

In some embodiments, to extract natural compounds efficiently, embodiments employ a supercritical fluid extraction method using carbon dioxide (CO₂) and ethanol as solvents. In some embodiments, the process begins with thorough drying and crushing of the selected plant materials. In some embodiments, following this preparation, the extraction process is conducted under precise conditions: a pressure of 30 Mpa, a temperature of 50 degrees Celsius, and a CO₂ flow rate of 5 mL/min. In some embodiments, this technique has been validated for the extraction of various compounds, including curcumin, terpenes, arnica metabolites, artemisinin, capsaicin, and Ginkgo biloba metabolites.

In some embodiments, after the extraction of the compounds from the plant species, the concentrates are filtered and then a rotary evaporator is used to separate the ethanol from each of the extracts. The substances are then lyophilized, and finally, the extracts will be purified.

In some embodiments, the nanoemulsion preparation utilizes high-energy methods like homogenization, ultrasonication or microfluidization to break down the cannabinoids and terpenes into nano-sized droplets. In some embodiments, this process improves their solubility in aqueous solutions and enhances bioavailability.

In some embodiments, the selection of carrier and stabilizers chooses appropriate carriers like: medium-chain triglycerides (MCT) oil and/or surfactants to stabilize the nanoemulsion and prevent droplet aggregation over time. In some embodiments, stabilizers like lecithin or polysorbates—may also be added to enhance stability. It should be noted that the selection of carriers, stabilizers, and surfactants is not limited to the options listed above, and other suitable agents may be used as required for the formulation.

In some embodiments, rigorous testing and characterization are conducted to evaluate the physical and chemical properties of the nanoemulsion. This includes assessing parameters such as particle size distribution, stability over time, and the encapsulation efficiency of cannabinoids and terpenes.

In some embodiments, bioavailability studies performed by in vitro and in vivo studies are used to assess the absorption and bioavailability of cannabinoids from the nanoemulsion formulation. In some embodiments, this includes evaluating how effectively the nanoemulsion delivers cannabinoids to target tissues, such as pancreatic cancer cells.

In some embodiments, high-energy nanoemulsification methods utilize mechanical devices to generate high-intensity cavitation in pre-mixed formulations, making them suitable for commercial nanoemulsion production. Cavitation induces high-shear forces that fragment and disperse oil droplets, forming stable nanoemulsions.

In some embodiments, High-Pressure Homogenization (HPH) refers to a group of nano-emulsification techniques that employ significant pressure differences to induce cavitation. In some embodiments, a HPH device applies high pressures to direct pre-emulsified formulations through an interaction chamber with microchannels. In some embodiments, these channels cause the formulations to collide, promoting cavitation.

In some embodiments, nanoemulsions are designed to enhance the bioavailability and therapeutic efficacy of cannabinoids for medical treatments. In some embodiments, in the context of pancreatic cancer, specifically for stages III, IV, and palliative care, the preparation of cannabinoid nanoemulsions involves creating a stable, finely dispersed, oil-in-water emulsion or water-in-oil emulsion.

In some embodiments, the methodologies used utilize high-energy processes, such as high-pressure homogenization, to reduce the oil droplet size and ensure consistent cannabinoid distribution. In some embodiments, the resulting nanoemulsion improves the solubility, stability, and absorption of cannabinoids, which possess anti-cancer properties targeting specific genes involved in cancer progression, such as KRAS and p53. In some embodiments, by effectively delivering these therapeutic compounds to the formulations disclosed inhibit tumor growth, induce apoptosis, antimetastatic activity, and improve the quality of life for patients with advanced pancreatic cancer.

In some embodiments, the preparation of a polyethylene glycol (PEG) cannabinoids nanoemulsion for therapeutic purposes, wherein PEG is a stabilizing solubilizing agent, specifically targeting pancreatitis treatment involves a meticulously designed process to ensure efficacy and safety. In some embodiments, the formulation begins with selecting cannabinoid compounds such as THC, THCV, CBD, CBG, and CBC, known for their potential therapeutic benefits in managing inflammation and pain associated with pancreatitis. In some embodiments, the formulation of cannabinoids is complemented by bioactive compounds such as limonene and kaempferol, which enhance their therapeutic effects.

In some embodiments, the emulsifying system utilizes emulsifying agents such as PEG-400, chosen for its lower molecular weight that enhances solubility in both water and organic solvents, facilitating the creation of a stable nanoemulsion with smaller droplet sizes. In some embodiments, medium-chain triglycerides (MCT oil) serve as the oil phase due to their superior solubility, stability, and compatibility with cannabinoid formulations. The aqueous phase consists of phosphate-buffered saline (PBS), providing stability, biocompatibility, and defined ionic strength essential for formulation consistency or purified water. Such emulsifying, solubilizing and solvent agents may comprise but not limited to Polyethylene Glycol, Glycerin, Propylene Glycol, Medium Chain Triglycerides (MCT oil), Mineral Oil, Sorbitol, Polysorbates (e.g., Tween 80), Sorbitan, Ethyl Alcohol (Ethanol), Oleic Acid, Lecithin, Sodium Lauryl Sulfate (SLS), Triacetin, and combinations thereof.

In some embodiments, during the preparation process, stabilizers such as Vitamin E are incorporated to protect the cannabinoids from degradation and improve their absorption and bioavailability. In some embodiments, equipment like high-shear homogenizers and ultrasonicators are employed to achieve uniform particle size distribution and enhance the stability of the nanoemulsion. In some embodiments, sterile filtration through a 0.22 μm filter ensures that the nanoemulsion is free from contaminants, meeting stringent quality and safety standards.

In some embodiments, the characterization of the nanoemulsion involves particle size analysis using specialized equipment to verify that droplet sizes or nanoparticle size are less than 200 nm, optimizing stability and bioavailability. In some embodiments, stability testing assesses parameters such as particle size, zeta potential, and cannabinoid concentration over time, to ensure consistent performance and efficacy throughout the product's shelf life.

In some embodiments, the capsule filling procedures are carefully executed using equipment calibrated for liquid formulations, ensuring precise dosing and uniformity across all capsules. Adherence to Good Manufacturing Practices (GMP) and rigorous safety and efficacy studies further validate the product's quality and suitability for therapeutic use in pancreatic cancer management. In some embodiments, this comprehensive approach to formulation and quality control aims to deliver a reliable and effective nanoemulsion product, potentially offering significant benefits to patients suffering from pancreatic disorders.

In some embodiments, before advancing to human trials, rigorous preclinical studies using cell cultures and animal models of pancreatic cancer were conducted to thoroughly evaluate the efficacy and safety of formulations described. In some embodiments, immunocompromised mice, specifically nude mice implanted with human pancreatic cancer cells (e.g., PANC-1), serve as models to simulate stage III and IV pancreatic cancer conditions to assess both its effectiveness in inhibiting tumor growth and safety parameters. In some embodiments, the study involves immunocompromised mice, aged 6-8 weeks, each receiving subcutaneous injections of 1×10\hat{ }6 PANC-1 cells into their right flank, with tumor growth monitored by caliper measurements every 3 days until tumors reach approximately 100 mm³. In some embodiments, a control group receives placebo nanoemulsion for baseline comparisons, while additional groups receive formulations described at varying concentrations to assess dose-response effects, administered via daily oral gavage throughout the study period.

In some embodiments, primary outcomes focus on evaluating tumor growth inhibition, supplemented by survival analysis and histopathological examinations to assess factors like necrosis, apoptosis, and treatment-related toxicity post-study.

In some embodiments, biomarker analysis of serum levels and gene expression studies (e.g., KRAS, p53) provide insights into physiological responses and cancer progression pathways affected by the formulations described. In some embodiments, safety evaluations include monitoring for weight changes, behavioral indicators, and comprehensive blood tests to assess liver and kidney function.

In some embodiments, trials employ a range of methods including imaging studies like CT scans and MRI to monitor tumor response and progression, biomarker analysis such as CA 19-9 levels to track disease markers, and clinical assessments to evaluate symptoms and quality of life. In some embodiments, measures like progression-free survival (PFS) and overall survival (OS) provide crucial endpoints to gauge treatment efficacy over time, comparing outcomes between treatment and control groups in randomized, double-blind studies.

In some embodiments, molecular analysis through biopsies and profiling further elucidates how formulations described influence tumor biology at a cellular level. In some embodiments, statistical analyses ensure robust interpretation of data, identifying significant treatment effects and supporting regulatory decisions.

Example embodiments of the claims are described. The described claims should not be considered limiting of the disclosure. In one embodiment, a method of treating metastatic or locally advanced pancreatic cancer in a human individual is disclosed. The method comprises administering to the human individual an effective concentration of a composition comprising nanoparticles comprising cannabinoids, terpenes and flavonoids (THC, THCV, CBD, CBG, CBD, limonene, and kaempferol) wherein the individual is selected for treatment based on at least one of (i) having stage III pancreatic cancer, (ii) having stage IV metastatic cancer (iii) having pancreatic cancer in a primary location in a pancreatic head, (iv) having more than 3 metastatic sites, and (v) having palliative care.

In another example embodiment, the method may be performed wherein the pancreatic cancer is pancreatic adenocarcinoma.

In another example embodiment, the method may be performed wherein the individual has biomarker results for CA19-9.

In another example embodiment, the method may be performed wherein the effective concentration of the composition in a PEG nanoemulsion composition is about 1270 mg of the principle active.

In another example embodiment, the method may involve administering one 1270 mg of the principle active capsule daily, for stage III patients over a consistent period of 6 to 10 weeks, and two capsules daily for stage IV patients over the same period. Preferably, the administration is consistent over an 8-week period for both stages, with stage IV patients taking two capsules daily.

In another example embodiment, the method may be performed wherein the nanoparticles in the composition have an average diameter of less than about 200 nm.

In another example embodiment, the method may be performed wherein the composition comprises a nanoemulsion capsule comprising THC, THCV, CBD, CBG, CBC, limonene, and kaempferol and the administration is oral.

In another example embodiment, the method may be performed wherein a percentage of each compound of cannabinoids, terpenes, and flavonoids for administration are THC-1.97 percent; THCV-3.15 percent; CBD-7.87 percent; CBG-6.29 percent; CBC-1.97 percent; limonene-39.37 percent; and kaempferol-39.37 percent.

In another example embodiment, the method may be performed wherein the administering of the effective concentration of the composition targets a head of the pancreas.

In another example embodiment, the method may be performed wherein the administering of the effective concentration of the composition targets adenocarcinoma in the pancreas.

In another example embodiment, a substance used to treat pancreatic cancer for a patient is disclosed, the substance comprising a composition comprising nanoparticles comprising cannabinoids, terpenes, and flavonoids (THC, THCV, CBD, CBG, CBD, limonene, and kaempferol) wherein a percentage of each compound of cannabinoids, terpenes, and flavonoids for administration are THC-1.97 percent; THCV-3.15 percent; CBD-7.87 percent; CBG-6.29 percent; CBC-1.97 percent; limonene-39.37 percent; and kaempferol-39.37 percent.

In another example embodiment, the substance may be configured in a capsule form.

In another example embodiment, the composition further comprises PEG-400 for a nanoemulsion of the nanoparticles. Preferably, the composition is a nanoemulsion formulation comprising nanoparticles.

In another example embodiment, the composition may be configured wherein the nanoemulsion with PEG-400 is configured to provide a controlled release profile of cannabinoids.

In another example embodiment, a substance used to treat pancreatic cancer for a patient is disclosed, the substance comprising a composition comprising nanoparticles comprising cannabinoids, terpenes, and flavonoids (THC, THCV, CBD, CBG, CBD, limonene, and kaempferol) wherein a percentage range of each compound of cannabinoids, terpenes, and flavonoids for administration are:

Formulation 1

| Component | Quantity (mg) | Percentage (%) |
|---|---|---|
| THC | 25 | 1.35 |
| THCV | 40 | 2.16 |
| CBD | 100 | 5.41 |
| CBG | 80 | 4.32 |
| CBC | 25 | 1.35 |
| Limonene | 500 | 27.03 |
| Kaempferol | 500 | 27.03 |
| Medium chain triglycerides | 120.4 | 6.51 |
| Sorbitan 80 (Span 80) | 40 | 2.16 |
| Vitamin E | 1.85 | 0.10 |
| Polysorbate 80 (Tween 80) | 40 | 2.16 |
| Polyethylene glycol 400 | 16 | 0.86 |
| PBS or purified water | 361.75 | 19.55 |

In another example embodiment, a substance used to treat pancreatic cancer for a patient is disclosed, the substance comprising a composition comprising nanoparticles comprising cannabinoids, terpenes, and flavonoids (THC, THCV, CBD, CBG, CBD, limonene, and kaempferol) wherein a percentage of each compound of cannabinoids, terpenes, and flavonoids for administration are:

Formulation 2

| Component | Quantity(mg) | Percentage (%) |
|---|---|---|
| THC | 25.9 | 1.4 |
| THCV | 40.7 | 2.2 |
| CBD | 101.75 | 5.5 |
| CBG | 83.25 | 4.5 |
| CBC | 25.9 | 1.4 |
| Limonene | 518 | 28 |
| Kaempferol | 518 | 28 |
| Medium chain triglycerides | 122.1 | 6.60 |
| Sorbitan 80 (Span 80) | 40.7 | 2.20 |
| Vitamin E | 3.7 | 0.2 |
| Polysorbate 80 (Tween 80) | 40.7 | 2.2 |
| Polyethylene glycol 400 | 18.5 | 1.00 |
| PBS or purified water | 310.8 | 16.8 |

Formulation 3

| Component | Quantity (mg) | Percentage (%) |
|---|---|---|
| THC | 18.5 | 1 |
| THCV | 27.75 | 1.5 |
| CBD | 83.25 | 4.5 |
| CBG | 64.75 | 3.5 |
| CBC | 18.5 | 1 |
| Limonene | 462.5 | 25 |
| Kaempferol | 462.5 | 25 |
| Medium chain triglycerides | 118.4 | 6.40 |
| Sorbitan 80 (Span 80) | 37 | 2.00 |
| Vitamin E | 1.665 | 0.09 |
| Polysorbate 80 (Tween 80) | 37 | 2.00 |
| Polyethylene glycol 400 | 12.95 | 0.7 |
| PBS or purified water | 505.3 | 25.31 |

In another example embodiment, the substance further comprises PEG-400 with the nanoemulsion.

In another example embodiment, the substance is configured wherein the nanoemulsion with PEG-400 is configured to provide a controlled release profile of cannabinoids.

In another example embodiment, a composition for treatment of pancreatic cancer is disclosed. The method may comprise an active principle; and a stabilizing solubilizing agent, wherein the active principle comprises a cannabinoid, a terpene, and a flavonoid.

In another example embodiment, the composition may be comprised wherein the active principle is present in a concentration in a range of 50% to 71%, and the stabilizing solubilizing agent is present in a concentration in a range of 0.1% to 5%.

In another example embodiment, the composition may be comprised wherein the cannabinoid is present in a concentration in a range of 10% to 15%, the terpene is present in a concentration in a range of 25% to 28%, and the flavonoid is present in a concentration in a range of 25% to 28%.

In another example embodiment, the composition may be comprised wherein the cannabinoid comprises THC, THCV, CBD, CBG, and CBC, the terpene comprises limonene and the flavonoid comprises kaempferol.

In another example embodiment, the composition may be comprised wherein the THC is present in a concentration in a range of 1% to 1.45%, the THCV is present in a concentration in the range of 1.5% to 2.25%, the CBD is present in a concentration in a range of 4.5% to 5.5%, the CBG is present in a concentration in the range of 3.5% to 4.5%, the CBC is present in a concentration in a range of 1% to 1.45%, the limonene is present in a concentration in a range of 25% to 28% and the kaempferol is present in a concentration in a range of 25% to 28%.

In another example embodiment, the composition may be comprised wherein the composition is a nanoemulsion formulation comprising nanoparticles.

In another example embodiment, the composition may be comprised wherein the stabilizing solubilizing agent is PEG-400.

In another example embodiment, the composition may be comprised wherein the nanoparticles have an average size of less than 200 nm.

In another example embodiment, the composition may be comprised wherein a nanoemulsion formulation is designed to provide a controlled release of the cannabinoids, the terpenes, and the flavonoids.

In another example embodiment, the composition may be comprised wherein the composition is suitable for oral administration in capsule form.

In another example embodiment, a method for treating one of metastatic and locally advanced pancreatic cancer in a human individual is disclosed. The method may comprise administering an effective concentration of a composition, wherein the composition comprises an active principle and a stabilizing solubilizing agent, wherein the active principle comprises a cannabinoid, a terpene, and a flavonoid.

In another example embodiment, the method may be performed wherein the human individual is selected for treatment based on at least one of: i) Stage Ill pancreatic cancer, ii) Stage IV metastatic pancreatic cancer, iii) Pancreatic cancer in the pancreatic head, iv) More than three metastatic sites, v) Patients under palliative care.

In another example embodiment, the method may be performed wherein the pancreatic cancer is pancreatic adenocarcinoma.

In another example embodiment, the method may be performed wherein the individual has biomarker results for CA19-9.

In another example embodiment, the method may be performed wherein the effective concentration is administered as one daily capsule containing 1270 mg of active principle for Stage III patients, and two daily capsules for Stage IV patients over an 8-week period.

In another example embodiment, the method may be performed wherein the effective concentration of active principle is in the range of 1143 mg to 1397 mg.

In another example embodiment, the method may be performed wherein the administering of the effective concentration of the composition of targets at least one of a pancreatic head and surrounding tissue.

In another example embodiment, a method for producing a pharmaceutical composition for treating pancreatic cancer, is disclosed. The method may comprise incorporating a cannabinoid, a terpene, and a flavonoid into a nanoplatform comprising a stabilizing solubilizing agent.

In another example embodiment, the method may be performed wherein the nanoplatform is a nanoemulsion comprising PEG-400 as a stabilizing solubilizing agent, in a form of nanoparticles.

In another example embodiment, the method may be performed wherein the nanoparticles have an average size of less than 200 nm.

In another example embodiment, the method may be performed wherein the formulation is tailored to provide a controlled release of the cannabinoids, terpenes, and flavonoids, and wherein the composition is administered in an oral dosage form selected from a group consisting of capsules, tablets, or liquid solutions.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

1. A composition for treatment of pancreatic cancer, comprising:
an active principle; and
a stabilizing solubilizing agent, wherein the active principle comprises a cannabinoid, a terpene, and a flavonoid.

2. The composition according to claim 1, wherein the active principle is present in a concentration in a range of 50% to 71%, and the stabilizing solubilizing agent is present in a concentration in a range of 0.1% to 5%.

3. The composition according to claim 2, wherein the cannabinoid is present in a concentration in a range of 10% to 15%, the terpene is present in a concentration in a range of 25% to 28%, and the flavonoid is present in a concentration in a range of 25% to 28%.

4. The composition according to claim 3, wherein the cannabinoid comprises THC, THCV, CBD, CBG, and CBC, the terpene comprises limonene and the flavonoid comprises kaempferol.

5. The composition according to claim 4, wherein the THC is present in a concentration in a range of 1% to 1.45%, the THCV is present in a concentration in the range of 1.5% to 2.25%, the CBD is present in a concentration in a range of 4.5% to 5.5%, the CBG is present in a concentration in the range of 3.5% to 4.5%, the CBC is present in a concentration in a range of 1% to 1.45%, the limonene is present in a concentration in a range of 25% to 28% and the kaempferol is present in a concentration in a range of 25% to 28%.

6. The composition according to claim 1, wherein the composition is a nanoemulsion formulation comprising nanoparticles.

7. The composition according to claim 1, wherein the stabilizing solubilizing agent is PEG-400.

8. The composition according to claim 1, wherein the nanoparticles have an average size of less than 200 nm.

9. The composition according to claim 1, wherein a nanoemulsion formulation is designed to provide a controlled release of the cannabinoids, the terpenes, and the flavonoids.

10. The composition according to claim 1, wherein the composition is suitable for oral administration in capsule form.

11. A method for treating one of metastatic and locally advanced pancreatic cancer in a human individual, comprising:
administering an effective concentration of a composition, wherein the composition comprises
an active principle and
a stabilizing solubilizing agent, wherein the active principle comprises a cannabinoid, a terpene, and a flavonoid.

12. The method according to claim 11, wherein the human individual is selected for treatment based on at least one of: i) Stage III pancreatic cancer, ii) Stage IV metastatic pancreatic cancer, iii) Pancreatic cancer in the pancreatic head, iv) More than three metastatic sites, v) Patients under palliative care.

13. The method according to claim 11, wherein the pancreatic cancer is pancreatic adenocarcinoma.

14. The method of claim 12, wherein the individual has biomarker results for CA19-9.

15. The method according to claim 11, wherein the effective concentration is administered as one daily capsule containing 1270 mg of active principle for Stage Ill patients, and two daily capsules for Stage IV patients over an 8-week period.

16. The method of claim 11, wherein the effective concentration of active principle is in the range of 1143 mg to 1397 mg.

17. The method according to claim 11, wherein the administering of the effective concentration of the composition of targets at least one of a pancreatic head and surrounding tissue.

18. A method for producing a pharmaceutical composition for treating pancreatic cancer, comprising: incorporating a cannabinoid, a terpene, and a flavonoid into a nanoplatform comprising a stabilizing solubilizing agent.

19. The method of claim 18, where the nanoplatform is a nanoemulsion comprising PEG-400 as a stabilizing solubilizing agent, in a form of nanoparticles.

20. The method according to claim 19, wherein the nanoparticles have an average size of less than 200 nm.

21. The method according to claim 20, wherein the formulation is tailored to provide a controlled release of the cannabinoids, terpenes, and flavonoids, and wherein the composition is administered in an oral dosage form selected from a group consisting of capsules, tablets, or liquid solutions.
